(19) United States
(12) Patent Application Publication
(10) Pub. No.: US 2025/0356485 A1
(43) Pub. Date: Nov. 20, 2025

(54) IMAGING FOR MAPPING A CLOTHED PERSON

(71) Applicant: Siemens Healthineers AG, Forchheim (DE)

(72) Inventors: Christopher SYBEN, Cadolzburg (DE); Christian HUEMMER, Lichtenfels (DE); Dominik ECKERT, Fuerth (DE)

(73) Assignee: Siemens Healthineers AG, Forchheim (DE)

(21) Appl. No.: 19/206,194

(22) Filed: May 13, 2025

(30) Foreign Application Priority Data
May 14, 2024 (DE) 10 2024 204 448.2

(51) Int. Cl. G06T 7/00

(52) U.S. Cl. CPC G06T 7/0012; G06T 2207/10088; G06T 2207/10116; G06T 2207/20081

(57) ABSTRACT

In a computer-implemented method for parameterizing an imaging system for mapping a clothed person, image data about the clothed person is obtained and body-shape information about the person is determined by applying a trained machine-learning model to the image data. At least one imaging parameter of the imaging system is determined as a function of the body-shape information.

### CROSS-REFERENCE TO RELATED APPLICATION(S)

The present application claims priority under 35 U.S.C. § 119 to German Patent Application No. 10 2024 204 448.2, filed May 14, 2024, the entire contents of which are incorporated herein by reference.

### FIELD

One or more example embodiments of the present invention relate to a computer-implemented method for parameterizing an imaging system for mapping a clothed person and an associated computer-implemented training method for a machine-learning model (MLM) for predicting body-shape information about a person on the basis of image data about the clothed person. One or more example embodiments of the present invention further relate to a data processing system for performing such computer-implemented methods or training methods, an imaging apparatus having such a data processing system, a corresponding imaging method for mapping a clothed person, and a corresponding computer program product.

### BACKGROUND

If medical images, such as X-ray images, MRT images or PET images, have to be taken of clothed persons, for example in emergencies or if it is not possible or desirable to undress the persons for other reasons, information about the body shape which is necessary or advantageous for configuring the imaging system is missing.

In current clinical practice, operators adjust the imaging system manually and estimate the body position or other information about the body shape by palpating the patient. It is conceivable for camera images of the person to be used for a rough pre-initialization of the imaging system. However, the patient's clothing, which cannot always be removed, in particular in trauma areas, prevents an accurate automated estimation of the patient's body-shape information.

The publication X. Zou et al.: “CLOTH4D: A Dataset for Clothed Human Reconstruction.”, Proceedings of the IEEE/CVE Conference on Computer Vision and Pattern Recognition 2023, proposes CLOTH4D, a dataset of clothed persons which contains 1,000 test subjects with different phenotypes, 1,000 3D outfits, and over 100,000 meshes for clothed people paired with unclothed people. By evaluating and retraining methods for reconstructing clothed people, new insights could thus be gained and performance improved.

The publication R. Vidaurre et al.: “Fully Convolutional Graph Neural Networks for Parametric Virtual Try-On”, Computer Graphics Forum, Proc. of ACM SIGGRAPH Symposium on Computer Animation 2020, proposes a learning-based approach for trying on clothing virtually, which is based on a convolutional neural graph network. This can handle a large family of clothing items, which are represented as parametric, predefined 2D panels with any mesh topology, including long dresses, shirts, and tight tops.

The publication H. Zhang et al.: “CloSET: Modeling Clothed Humans on Continuous Surface with Explicit Template Decomposition.”, Proceedings of the IEEE/CVF Conference on Computer Vision and Pattern Recognition 2023, describes the creation of animatable avatars from static scans. This requires the modeling of deformations of clothing in different poses. To this end, point-based solutions are addressed and it is proposed to deconstruct explicit clothing-related templates and then add position-dependent convolutions to them.

The publication J. Zhu et al.: “Unpaired image-to-image translation using cycle-consistent adversarial networks.”, Proceedings of the IEEE international conference on computer vision 2017, proposes an approach to image-to-image translation, which deals with a class of image processing and graphics problems in which the goal is to learn the association between an input image and an output image using a training set of matched image pairs. In accordance with the proposed approach, it is possible to learn to translate an image from a source domain X to a target domain Y in the absence of paired samples. In this case an adversarial loss is used to learn a mapping G: X→Y, so that the distribution of the images from G(X) cannot be distinguished from the distribution Y.

The Unity Engine (https://github.com/nielsdos/UnityClothSimulation.git, retrieved on Apr. 22, 2024) is simulation software for simulating clothing fabrics. The Unreal Engine uses the Chaos Cloth Solver to simulate clothing

(https://dev.epicgames.com/documentation/en-us/unreal-engine/clothing-tool-in-unreal-engine?application_version=5.2, retrieved on Apr. 22, 2024).

### SUMMARY

It is an object of one or more embodiments of the present invention to estimate body-shape information about a clothed person automatically with a higher degree of accuracy.

At least this object is achieved by the respective subject matter of the independent claims. Advantageous developments and preferred forms of embodiment are the subject matter of the dependent claims.

One or more example embodiments of the present invention are based on the idea of determining body-shape information about a person, in particular in the unclothed state, on the basis of image data about the person in the clothed state via a correspondingly trained machine-learning model.

In accordance with one aspect of embodiments of the present invention a computer-implemented method for parameterizing an imaging system for mapping a clothed person is specified. In this case, image data about the clothed person is obtained and body-shape information about the person is determined by applying a trained machine-learning model (MLM) to the image data. At least one imaging parameter of the imaging system is determined as a function of the body-shape information.

Unless specified otherwise, all steps of the computer-implemented method can be performed by a data processing system which contains at least one data processing device. In particular, the at least one data processing device is designed or adapted to execute the steps of the computer-implemented method. For this purpose the at least one data processing device can for example store a computer program which contains commands which, if they are executed by the at least one data processing device, cause the at least one data processing device to execute the computer-implemented method. The computer-implemented method can also be implemented wholly or partially in the hardware. The expressions “data processing system” and “at least one data processing device” can be used interchangeably here and below. This also applies for corresponding expressions derived therefrom.

If the at least one data processing device contains two or more data processing devices, certain steps performed by the at least one data processing device can also be understood to mean that different data processing devices perform different steps or different parts of a step. In particular, it is not necessary for every data processing device to perform the steps. In other words, the performance of the steps can be distributed to the two or more data processing devices.

Each form of embodiment of the computer-implemented method results in a corresponding form of embodiment of a method for parameterizing an imaging system which is not purely computer-implemented by including corresponding steps for generating the image data.

In general terms, a trained MLM can mimic cognitive functions which humans associate with another human mind. In particular, the MLM is able, thanks to training on the basis of training data, to adjust itself to new circumstances and to detect and extrapolate patterns. Another term for a trained MLM is “trained function”.

In general, the parameters of an MLM can be adjusted or updated by training. In this case, supervised training, semi-supervised training, unsupervised training, reinforcement learning and/or active learning can be used in particular. In addition, use can be made of representation learning, also known as feature learning. In particular, the parameters of the MLMs can be adjusted iteratively by multiple steps of the training. In particular, training can minimize a certain loss function, which is also known as the cost function. When training an artificial neural network (ANN) the backpropagation algorithm can be used in particular.

An MLM can in particular include an ANN, a support vector machine, a decision tree and/or a Bayesian network, and/or the MLM can be based on k-means clustering, Q-learning, genetic algorithms and/or association rules. In particular, an ANN can be or can include a deep neural network, a convolutional neural network (CNN) or a convolutional deep neural network. In addition, an ANN can be an adversarial network, a deep adversarial network and/or a generative adversarial network (GAN).

In the present case, the MLM is trained such that it can predict the body-shape information on the basis of input data dependent on the image data, or that it can predict an output on the basis of the input data dependent on the image data, from which body-shape information can be derived directly, for example a corresponding body model. The input data can in this case include the image data or can be calculated on the basis of the image data. For example, the input data can be calculated by encoding the image data, for example via a further trained MLM. The input data is then given by the encoded image data.

The image data maps the person who is in particular to be mapped by the imaging system in the clothed state, so that in particular the person's body shape is not or not completely visible. The body-shape information relates to the body shape of the same person in the unclothed state.

The term “parameterization” can in particular be understood to mean that the at least one imaging parameter is determined, thus that a corresponding value is determined for each imaging parameter of the at least one imaging parameter. Which parameters are involved in the case of the at least one imaging parameter is in particular predefined and depends on the specific application, thus in particular on the type of imaging system and where appropriate the type of imaging method to be performed therewith.

For example, the imaging system is an X-ray-based imaging system, for example an X-ray angiography system, a C-arm X-ray system, an X-ray tomosynthesis system or a computed tomography system. The at least one imaging parameter can then for example contain at least one exposure setting of an X-ray source of the imaging system and/or a detector amplification of an X-ray detector of the imaging system and/or a collimator position of a collimator of the imaging system and/or a size of a collimator aperture of the collimator and/or a shape of a collimator aperture of the collimator.

The at least one exposure setting can for example contain a peak kilovoltage (kVp) and/or a tube current of the X-ray source and/or a pulse duration of the X-ray pulses emitted by the X-ray source.

In other exemplary applications, the imaging system is a magnetic resonance tomography system and the at least one imaging parameter contains a mapping target domain within a patient tube of the magnetic resonance tomography system. The same applies for example to other imaging systems, for instance positron emission tomography systems.

If the at least one imaging parameter was determined as a result of the inventive computer-implemented method, the imaging system can be configured in accordance with the specified at least one imaging parameter. The clothed person can then be mapped via the imaging system configured in this way.

In accordance with embodiments of the present invention the correspondingly trained MLM is thus used to predict the person's body-shape information which cannot be directly identified by a human observer from the image data, and as a function thereof to derive the corresponding imaging parameters of the imaging system. It is thus no longer necessary to palpate the person's body manually in order to be able to estimate the person's body shape approximately. Furthermore, the MLM provides more accurate results, which leads to a better determination of the imaging parameters and ultimately to a better quality of the results of the mapping. It has been shown that in particular ANNs, for example GANs, CNNs and transformer networks, are particularly suitable as MLMs in the present case.

Using the inventively determined at least one imaging parameter, the imaging system can then be configured, in particular automatically or partially automatically, in accordance with the determined at least one imaging parameter. Thus the operation of the imaging system can be further automated.

In accordance with at least one form of embodiment the image data contains a two-dimensional or two-and-a-half-dimensional image, in particular a camera image, of the clothed person.

The image of the clothed persons can thus be recorded via a corresponding camera if the person is to be mapped with the imaging system. Accordingly, the generation of image data can easily be integrated into the clinical procedure. On the other hand, it is known, for example from the publications explained in the introduction, that camera images are very well suited for the corresponding projection via MLMs.

A two-dimensional image maps a scene two-dimensionally. An associated intensity value is accordingly present for each pixel of a two-dimensional arrangement of pixels. A two-dimensional image can however also have multiple channels, in particular color channels. A two-and-a-half-dimensional image on the other hand also contains a depth value for each pixel in addition to the intensity value, which indicates the distance of the corresponding pixel in the scene from the camera. Such images can for example be generated with ToF cameras (TOF: “Time of Flight”) or flash lidar systems. In the present context, two-and-a-half-dimensional images have the advantage that the additional depth information available makes possible a more accurate prediction or calculation of the body-shape information.

In accordance with at least one form of embodiment the image data contains a video of the clothed person.

In other words the image data contains a sequence of consecutive two-dimensional or two-and-a-half-dimensional camera images. The camera images of the sequence show the clothed person in this case in particular from different viewing directions. The image data effectively thus contains three-dimensional image information about the clothed person. Thus a more accurate prediction or calculation of the body-shape information becomes possible.

In accordance with at least one form of embodiment the image data contains a two-and-a-half-dimensional or three-dimensional point cloud which represents the clothed person.

Such point clouds can for example be generated with laser scanners. In a two-and-a-half-dimensional point cloud the point cloud contains a two-dimensional position and a distance or depth for each point, similarly as described above for two-and-a-half-dimensional images. In a two-and-a-half-dimensional point cloud the viewing direction is in this case fixed. A three-dimensional point cloud contains this information for different viewing directions, similarly to the case of a video containing images from different viewing directions. Thus a more accurate prediction or calculation of the body-shape information becomes possible.

In accordance with at least one form of embodiment the body-shape information describes a body contour of the person, in particular in the unclothed state.

In other words the body-shape information provides information about where the person's body begins or ends, which because of the clothing is not or not reliably identifiable from the image data for operators of the imaging system or the like. However, the body contour can have a significant influence on the choice of the at least one imaging parameter, so that such forms of embodiment are particularly advantageous.

In accordance with at least one form of embodiment the body-shape information specifies respective positions of characteristic points of the person's body.

The characteristic points, also referred to as keypoints, are for example predefined points on the person's body, for instance joints, for example shoulder joints, elbow joints, knee joints, hip joints, wrists, ankles, etc. Other examples of characteristic points are the solar plexus, defined points on the person's head or face, etc.

The position of the characteristic points can be used to determine the at least one imaging parameter, but because of the clothing may not or not reliably be identifiable from the image data for operators or the like. Hence such forms of embodiment are particularly advantageous.

In accordance with at least one form of embodiment secondary body information about the person is estimated as a function of the body-shape information and the at least one imaging parameter is determined as a function of the secondary body information.

The secondary body information relates in particular to the internal nature of the body. Although this is not body-shape information, it can be determined at least approximately as a function thereof, which is why it is referred to here and below as secondary. Such secondary body information can have a significant influence on the choice of the at least one imaging parameter, so that such forms of embodiment are particularly advantageous.

For example, the secondary body information can contain the person's body weight and/or a material composition of the person's body. The material composition can for example be the mass ratio or volume ratio of a material in the person's body to another material in the person's body, for instance of bone tissue to fat tissue, water to fat tissue, muscle tissue to fat tissue, etc. The material composition can also be a body fat percentage, a muscle mass, or the like.

In accordance with at least one form of embodiment a body model of the person is generated by applying the MLM to the image data, and the body-shape information is determined as a function of the body model.

As explained in the introduction, known models exist, which from a person's body model can predict what the person would look like clothed, including for different clothing variants. In the present forms of embodiment of the inventive computer-implemented method, this approach is reversed to a certain extent, so that the body model is predicted from the clothed person. The body-shape information can in turn be extracted directly from the body model and/or the secondary body information can be determined. Such embodiments are in particular advantageous, since once a body model is known, different body-shape information and/or secondary body information can also be determined for different imaging methods as required, and the MLM does not have to be trained anew for this in each case.

In accordance with a further aspect of embodiments of the present invention a computer-implemented training method for an MLM, in particular an ANN, is specified for the prediction of body-shape information about a person on the basis of image data about the clothed person, in particular for use in an inventive computer-implemented method. In this case training data is obtained and the untrained or partially trained MLM is trained as a function of the training data, supervised or unsupervised, to apply the MLM to the image data to predict the body-shape information or to predict a body model of the person, from which the body models can be derived.

In accordance with at least one form of embodiment the MLM contains a convolutional neural network (CNN) or a transformer network, for example a vision transformer network.

The training takes place for example unsupervised. In this case the training data contains a variety of training datasets. Each of the training datasets contains training image data about a clothed person and associated basic truth data.

The same applies for the training image data for example, as was stated above for the image data. It can in particular in each case contain a two-dimensional or two-and-a-half-dimensional image of the clothed person and/or a video of the clothed person and/or a two-and-a-half-dimensional or three-dimensional point cloud. However, it can also relate to corresponding simulated images, videos or point clouds, or to images, videos or point clouds of clothed phantom objects or the like.

The associated basic truth data of a training image dataset contains the associated body-shape information or the associated body model for the person or simulated person or the phantom object which is mapped by the training image data of the same training image dataset. Thus there is a one-to-one assignment of the training image data and the basic truth data of each training dataset. In other words each training dataset contains a pair of datasets associated with one another, namely the respective training image data and the associated basic truth data.

As already mentioned, such pairs of datasets assigned to one another can be generated by simulation. For example, graphics engines can be used for this purpose, as are also used for computer games, for example the Unreal Engine mentioned in the introduction or the aforementioned Unity Engine.

In addition, known methods for supervised training can in particular be used to train the CNN or transformer network.

In accordance with at least one form of embodiment the MLM contains a generative adversarial network (GAN).

The GAN can contain at least one discriminator and at least one generator. After the training, a generator of the GAN is able to predict the body model or the body-shape information on the basis of the image data. After the training only this generator and not the entire GAN is where appropriate then necessary for performing an inventive computer-implemented method.

For example, the training takes place unsupervised. The training data contains a variety of first training datasets. Each of the first training datasets contains training image data about a clothed person. The training data contains a variety of second training datasets, wherein each of the second training datasets contains training body-shape information or a training body model of a person.

However, in this case the training image data of the first training datasets and the training body-shape information or training body models of the second training datasets do not need to be assigned to one another, unlike in the case of supervised training. It is therefore not necessary to provide associated pairs, as described above for the supervised training. In addition, however, the training data can be generated in a similar manner.

In addition, known methods for supervised training can in particular be used to train the GAN. For example, the GAN can be designed in accordance with the cycle-consistent adversarial networks mentioned in the introduction.

In accordance with a further aspect of embodiments of the present invention an imaging method for mapping a clothed person is specified. In this case at least one imaging parameter of an imaging system is determined, by performing an inventive computer-implemented method. The clothed person is mapped via the imaging system configured in accordance with the at least one imaging parameter.

Further forms of embodiment of the inventive imaging method follow on directly from the various embodiments of the inventive computer-implemented method and of the inventive computer-implemented training method and vice versa.

In accordance with a further aspect of embodiments of the present invention a data processing system is specified, which is adapted to perform an inventive computer-implemented method for parameterizing an imaging system.

In accordance with a further aspect of embodiments of the present invention a further data processing system is specified, which is adapted to perform an inventive computer-implemented training method.

In the present disclosure the expressions “data processing system” and “at least one data processing device” can be used interchangeably. A data processing device can in particular be understood to mean a data processing device which contains a processing circuit. The data processing device can thus in particular process data for the performance of arithmetical operations. Where appropriate this also includes operations to perform indexed accesses to a data structure, for example a look-up table (LUT), as well as a data processing process implemented in hardware.

The data processing device can in particular contain one or more computers, one or more microcontrollers and/or one or more integrated circuits, for example one or more application-specific integrated circuits (ASIC), one or more field-programmable gate arrays (FPGA) and/or one or more systems on a chip (SoC). The data processing device can also contain one or more processors, for example one or more microprocessors, one or more central processing units (CPU), one or more graphics processing units (GPU) and/or one or more signal processors, in particular one or more digital signal processors (DSP). The data processing device can also contain a physical or virtual network of computers or other of the aforementioned units.

In different exemplary embodiments the data processing device contains one or more hardware and/or software interfaces and/or one or more memory units.

A memory unit can be designed as a volatile data memory, for example as a dynamic random access memory (DRAM) or static random access memory (SRAM), or as a nonvolatile data memory, for example as a read-only memory (ROM), as a programmable read-only memory (PROM), as an erasable programmable read-only memory (EPROM), as an electrically erasable programmable read-only memory (EEPROM), as a flash memory or flash EEPROM, as a ferroelectric random access memory (FRAM), as a magnetoresistive random access memory (MRAM) or as a phase-change random access memory (PCRAM).

In accordance with a further aspect of embodiments of the present invention an imaging apparatus is specified. The imaging apparatus has an imaging system and a data processing system. The data processing system is adapted to perform an inventive computer-implemented method for parameterizing an imaging system, in order to determine at least one imaging parameter of the imaging system. The imaging system is designed to map a clothed person in accordance with the at least one imaging parameter.

In accordance with a further aspect of embodiments of the present invention a first computer program containing first commands is specified. If the first commands are executed by a data processing system, the commands cause the data processing system to perform an inventive computer-implemented method for parameterizing an imaging system.

In accordance with a further aspect of embodiments of the present invention a second computer program containing second commands is specified. If the second commands are executed by a data processing system, the commands cause the data processing system to perform an inventive computer-implemented training method.

In accordance with a further aspect of embodiments of the present invention a third computer program containing third commands is specified. If the third commands are executed by an inventive imaging apparatus, in particular the data processing system of the imaging apparatus, the commands cause the imaging apparatus to perform an inventive imaging method.

The first commands, the second commands and/or the third commands can for example each be present as program code. The program code can for example be provided as binary code or Assembler and/or as source code of a programming language, for example C, and/or as a program script, for example Python.

In accordance with a further aspect of embodiments of the present invention a computer-readable storage medium is specified, which stores an inventive first computer program and/or an inventive second computer program and/or an inventive third computer program.

The first computer program, the second computer program, the third computer program, and the computer-readable storage medium are each computer program products containing the first commands, the second commands and/or the third commands.

Above and below, the inventive solution is described both in relation to the claimed systems and in relation to the claimed methods. Features, advantages or alternative forms of embodiment can be assigned to the other claimed subject matters and vice versa. In other words, the claims and forms of embodiment for the systems can be improved by features which are described or claimed in connection with the respective methods. In this case, the functional features of the method are implemented by physical units of the system.

In addition, the inventive solution is described above and below in relation to methods and systems for parameterizing an imaging system as well as in relation to methods and systems for providing a trained MLM. Features, advantages or alternative forms of embodiment can be assigned to the other claimed subject matters and vice versa. In other words, claims and forms of embodiment for providing a trained MLM can be improved with features which are described or claimed in connection with the parameterization of an imaging system. In particular, the datasets used in the methods and systems can have the same properties and features as the corresponding datasets which are used in the methods and systems for providing a trained MLM, and the trained MLMs provided by the respective methods and systems can be used in the methods and systems for parameterizing an imaging system.

Further features and combinations of features of embodiments of the present invention emerge from the figures and the description thereof as well as from the claims. In particular, further forms of embodiments of the present invention need not necessarily contain all features of one of the claims. Further forms of embodiments of the present invention can have features or combinations of features which are not mentioned in the claims.

### BRIEF DESCRIPTION OF THE DRAWINGS

The present invention is explained in greater detail below using specific exemplary embodiments and associated schematic drawings. In the figures, identical or functionally identical elements can be provided with the same reference characters. The description of identical or functionally identical elements is where appropriate not necessarily repeated in respect of different figures.

In the figures:

FIG. 1 shows a schematic representation of an exemplary form of embodiment of an inventive imaging apparatus;

FIG. 2 shows a schematic flow diagram of an exemplary form of embodiment of an inventive computer-implemented method for parameterizing an imaging system;

FIG. 3 shows a schematic flow diagram of a further exemplary form of embodiment of an inventive computer-implemented method for parameterizing an imaging system;

FIG. 4 shows a schematic flow diagram of an exemplary form of embodiment of an inventive computer-implemented training method;

FIG. 5 shows a schematic flow diagram of a further exemplary form of embodiment of an inventive computer-implemented training method;

FIG. 6 shows a schematic flow diagram of a further exemplary form of embodiment of an inventive computer-implemented training method;

FIG. 7 schematically shows a CNN; and

FIG. 8 schematically shows an ANN.

### DETAILED DESCRIPTION

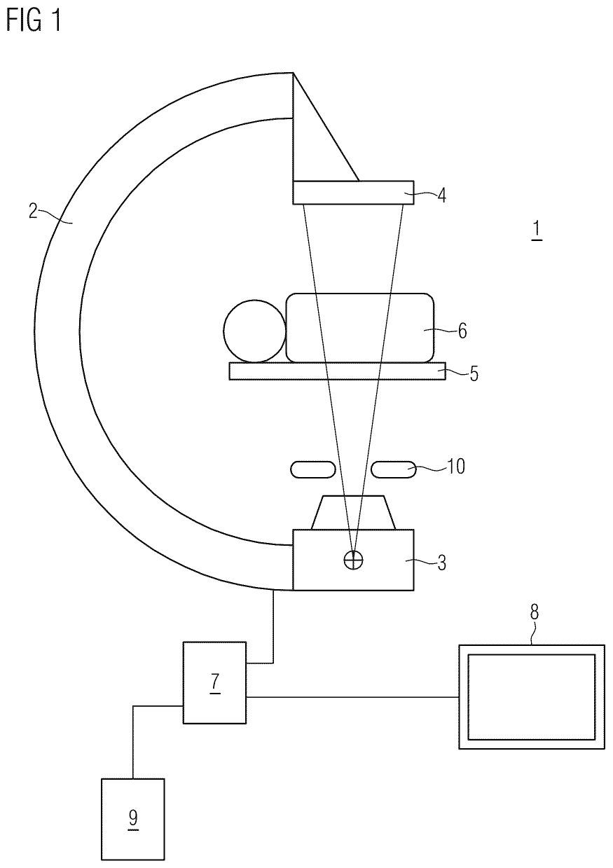

FIG. 1 shows a schematic illustration of an exemplary form of embodiment of an inventive imaging apparatus 1. The imaging apparatus 1 has an imaging system and a data processing system 7, 9.

In the example in FIG. 1, the imaging system is an X-ray imaging system and comprises a source unit 3 containing an X-ray source, a detector unit 4 containing an X-ray detector, and a control system 7 which is designed to control the X-ray source and the X-ray detector, in order to generate X-ray images which map a clothed person 6.

The X-ray imaging system can for example comprise a patient table 5 on which the object 6 is arranged. The X-ray imaging system 1 comprises an inventive data processing system 9. Some functions and method steps which are executed by the control system 7 are described below, while other functions and method steps are described which are executed by the data processing system 9. It should be noted that the functions and method steps can also be distributed differently in alternative forms of embodiment.

Thus the control system 7 can for example set different imaging parameters of the X-ray imaging system, including for example exposure parameters such as a peak kilovoltage of the X-ray source, a tube current of the X-ray source and/or an X-ray pulse duration. The control system 7 can for example set further imaging parameters such as the filter material and/or the filter thickness of an X-ray filter, for example a copper filter, by introducing the corresponding X-ray filter into the beam path or removing it from the beam path. The control system 7 can for example set further imaging parameters, such as the size of the collimator aperture of an X-ray collimator 10. For example, the control system 7 can bring the X-ray collimator 10 into the beam path or remove it from the beam path. The control system 7 can for example set further imaging parameters such as an amplification factor of the X-ray detector. Thus the control system 7 can for example bring an anti-scattering grid into the beam path or remove it from the beam path.

In some implementations, the X-ray imaging system comprises a display apparatus 8, wherein the control system 7 is designed to control the display apparatus 8, in order to display X-ray images or processed X-ray images.

In some implementations, the X-ray imaging system 1 is embodied as a fluoroscopy system, in particular as a C-arm fluoroscopy system, in which the source unit 3 and the detector unit 4 are mounted opposite one another on a C-arm 2 which can be rotated about different axes. The corresponding movements are referred to as angular or orbital movement. In some embodiments, the patient table 5 and the C-arm 2 can be positioned relative to one another by corresponding translational movements of the C-arm 2 and/or of the patient table 5, apart from the rotational movement of the C-arm 2. Consequently, the position and/or the orientation of the X-ray source with respect to the object 6 and the position and/or the orientation of the X-ray detector with respect to the object 6 can be set precisely to the desired image perspective.

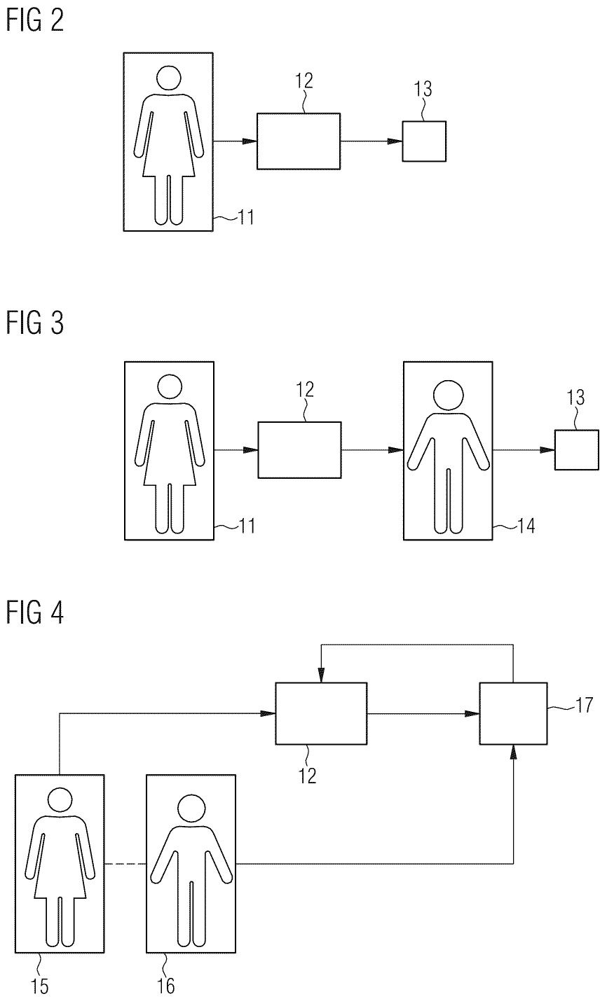

The data processing system 7, 9 is adapted to perform an inventive computer-implemented method for parameterizing the imaging system, in order to determine at least one imaging parameter 13 of the imaging system, as represented schematically in FIG. 2 and FIG. 3. The imaging system is designed, after it has been configured correspondingly in accordance with the at least one imaging parameter 13, to map a clothed person 6 in accordance with the at least one imaging parameter 13.

In accordance with the computer-implemented method for parameterizing the imaging system, image data 11 about the clothed person 6 is obtained, for example a two-dimensional or two-and-a-half-dimensional image or a video of the clothed person 6. Body-shape information about the person 6 is determined, for example a body contour of the person 6 and/or respective positions of characteristic points of the body of the person 6, by applying a trained MLM 12 to the image data 11. In the example in FIG. 2, the MLM 12 is correspondingly trained to predict the body-shape information directly on the basis of the image data 11. In the example in FIG. 3, the MLM 12 is trained to generate a body model 14 of the person 6 and the body-shape information is then determined as a function of the body model 14. In both cases the at least one imaging parameter 13 of the imaging system is determined as a function of the body-shape information.

FIG. 4 to FIG. 6 schematically show flow diagrams of respective exemplary forms of embodiment of inventive computer-implemented training methods for an MLM 12 for use in an inventive computer-implemented method for parameterizing an imaging system, as described for example in respect of FIG. 1 to FIG. 3.

In this case training data is obtained and the untrained or partially trained MLM 12 is trained as a function of the training data, supervised or unsupervised, to predict the body-shape information by applying the MLM 12 to the image data 11 or to predict the body model 14 of the person, from which the body-shape information can be derived.

In the example in FIG. 4 the MLM 12 for example contains a CNN and the training is supervised. In particular, the training data contains a variety of training datasets, wherein each of the training datasets contains training image data 15 about a clothed person 6 and associated basic truth data 16. An evaluation block 17 calculates a loss function on the basis of a deviation of the output predicted by the MLM 12 on the basis of the training image data 15. Based on the result, the MLM 12 is updated, for example using backpropagation.

In this way the MLM 12 can for example be trained to output an image of the person 6, in which the body contour is highlighted. It is also possible for the MLM 12 to output the positioning of characteristic points on the body of the person 6 and/or secondary body information about the person 6, such as weight or height.

In the example in FIG. 5, the MLM 12 is for example a generator module of a GAN 21 and the training is unsupervised. The training data contains a variety of first training datasets, wherein each of the first training datasets contains training image data 18 about a clothed person. The training data contains a variety of second training datasets, wherein each of the second training datasets contains training body-shape information or a training body model 19 of a person. An evaluation block 20 calculates one or more loss functions, as is known per se for GANs 21.

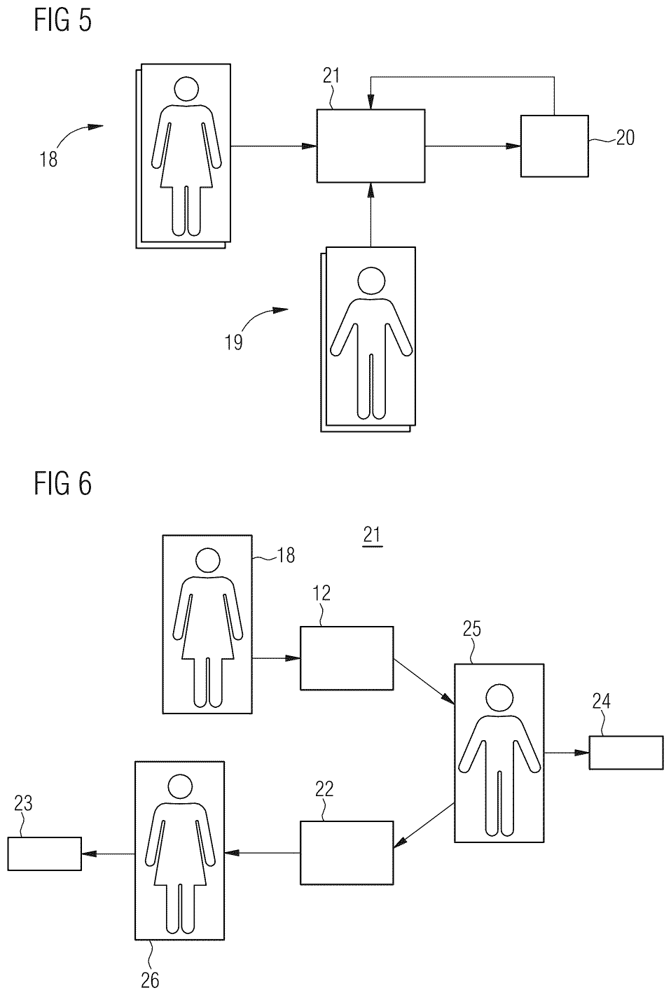

The example in FIG. 6 is based on that in FIG. 5. The GAN 21 is in this case designed as a cycle-consistent adversarial network. The GAN 21 has a first generator 12, which predicts a body model 25 on the basis of the training image data 18 for example. A first discriminator 24 assesses whether the predicted body model 25 matches the distribution of the training body model 19. A second generator 22 in turn predicts image data 26 about the clothed person 6 on the basis of the predicted body model 25. A second discriminator 23 assesses whether the predicted image data 26 matches the distribution of the training image data 18. The training of the generators 12, 22 and discriminators 23, 24 can take place in a known manner, in particular in a graduated manner, as described for instance in the publication by J. Zhu et al. mentioned in the

### INTRODUCTION

Once the training is concluded, the first generator 12 is able correctly to predict the body model 14 on the basis of the image data 11 about the clothed person 6 and can thus be used as an MLM 12 as described in respect of FIG. 3.

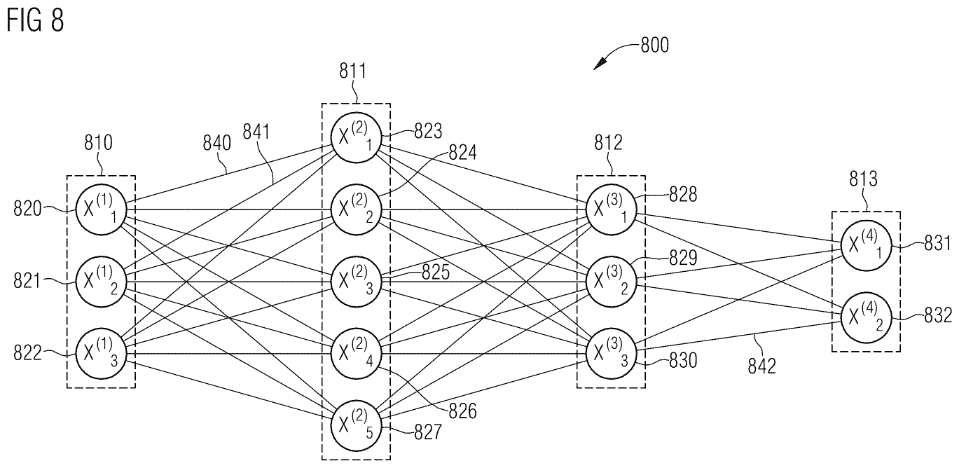

FIG. 8 shows a form of embodiment of an artificial neural network (ANN) 800. The ANN 800 comprises nodes 820, . . . , 832 and edges 840, . . . , 842, wherein each edge 840, . . . , 842 is a directed connection from a first node 820, . . . , 832 to a second node 820, . . . , 832. In general the first node 820, . . . , 832 and the second node 820, . . . , 832 are different nodes 820, . . . , 832. However, it is also possible for the first node 820, . . . , 832 and the second node 820, . . . , 832 to be identical. In FIG. 8 for example, the edge 840 is a directed connection from the node 820 to the node 823 and the edge 842 is a directed connection from the node 830 to the node 832. An edge 840, . . . , 842 from a first node 820, . . . , 832 to a second node 820, . . . , 832 is also referred to as an incoming edge for the second node 820, . . . , 832 and as an outgoing edge for the first node 820, . . . , 832.

In this example the nodes 820, . . . , 832 of the ANN 800 can be arranged in layers 810, . . . , 813, wherein the layers can have an intrinsic order, which is introduced by the edges 840, . . . , 842 between the nodes 820, . . . , 832. In particular, the edges 840, . . . , 842 can exist only between adjacent layers of nodes. In the example shown there is an input layer 810 which consists only of the nodes 820, . . . , 822 without incoming edges, an output layer 813 which consists only of the nodes 831, 832 without outgoing edges, and hidden layers 811, 812 between the input layer 810 and the output layer 813. In general, the number of hidden layers 811, 812 can be selected arbitrarily. In a multilayer perceptron (MLP) this number is at least one. The number of nodes 820, . . . , 822 within the input layer 810 generally relates to the number of input values of the artificial neural network 800, and the number of nodes 831, 832 within the output layer 813 generally relates to the number of output values of the artificial neural network 800.

In particular, each node 820, . . . , 832 of the artificial neural network 800 can be assigned a real number as a value. In this case x(n) i refers to the value of the i-th node 820, . . . , 832 of the n-th layer 810, . . . , 813. The values of the nodes 820, . . . , 822 of the input layer 810 correspond to the input values of the artificial neural network 800. The values of the nodes 831, 832 of the output layer 813 correspond to the output value of the artificial neural network 800. Additionally, each edge 840, . . . , 842 can have a weight, which is a real number. In particular, the weight is a real number within the interval [−1, 1] or within the interval [0, 1]. In this case w(m, n) i, j refers to the weight of the edge between the i-th node 820, . . . , 832 of the m-th layer 810, . . . , 813 and the j-th node 820, . . . , 832 of the n-th layer 810, . . . , 813. Additionally, the abbreviation w(n)i, j for the weight w(n, n+1)i, j is defined. In order to calculate the output values of the neural network 800, the input values are in particular propagated by the neural network 800. In particular, the values of the nodes 820, . . . , 832 of the (n+1)-th layer 810, . . . , 813 can be calculated on the basis of the values of the nodes 820, . . . , 832 of the n-th layer 810, . . . , 813 as

$${x}_{j}^{( n + 1 )} = f ( {\sum }_{i} {x}_{i}^{( n )} {w}_{i , j}^{( n )} ) .$$

In this, the function f is referred to as the transfer function or activation function. Well-known transfer functions are step functions, sigmoid functions, for example the logistical function, the generalized logistical function, the hyperbolic tangent, the arc tangent function, the error function, the smooth step function or rectifier functions. The transfer function is for example used for normalization. In particular, the values are propagated layer by layer by the neural network 800, wherein the values of the input layer 810 are given by the input of the neural network 800, wherein the values of the first hidden layer 811 can be calculated on the basis of the values of input layer 810 of the neural network 800, wherein the values of the second hidden layer 812 can be calculated on the basis of the values of the first hidden layer 811, etc.

To establish the values w(m, n)i, j for the edges, the neural network 800 must be trained using training data. The training data in particular comprises training input data and training output data (referred to as ti). In a training step the neural network 800 is applied to the training input data in order to generate calculated output data. In particular, the training data and the calculated output data comprises a number of values, corresponding to the number of nodes of the output layer. In particular, a comparison between the calculated output data and the training data is used to adjust the weights within the neural network 800 recursively (backpropagation algorithm). In particular, the weights are changed in accordance with the following formula

$${w}_{i , j}^{′ ( n )} = {w}_{i , j}^{( n )} - γ {δ}_{j}^{( n )} {x}_{i}^{( n )} ,$$

where γ is a predefined learning rate, and the numbers δ(n)j can be recursively calculated as

$${δ}_{j}^{( n )} = ( {\sum }_{k} {δ}_{k}^{( n + 1 )} {w}_{j , k}^{( n + 1 )} ) {f}^{′} ( {x}_{i}^{( n )} {w}_{i , j}^{( n )} )$$

on the basis of δ(n+1)j, if the (n+1)-th layer is not the output layer 813, and

$${δ}_{j}^{( n )} = ( {x}_{j}^{( n + 1 )} - {t}_{j}^{( n + 1 )} ) {f}^{′} ( {x}_{i}^{( n )} {w}_{i , j}^{( n )} ) ,$$

if the (n+1)-th layer is the output layer 813, where f′ is the first derivation of the activation function, and t(n+1) j is the comparison training value for the j-th node of the output layer 813.

A convolutional neural network (CNN) is an ANN which in at least one of its layers uses a convolution operation instead of a general matrix multiplication. These layers are referred to as convolution layers. In particular, a convolution layer performs a point product of one or more convolution kernels with the input data of the convolution layer, wherein the entries of the one or more convolution kernels are parameters or weights which can be adjusted by training. In particular, use can be made of the internal Frobenius product and the ReLU activation function. A convolutional neural network can comprise additional layers, for example pooling layers, fully connected layers, and/or normalization layers.

By using convolutional neural networks, the input can be processed very efficiently, since a convolution operation which is based on different kernels can extract different image features, so that by adjusting the weights of the convolutional kernel the relevant image features can be determined during the training. Additionally, due to the shared use of the weights in the convolution kernels, fewer parameters need to be trained, which prevents any over-adjustment in the training phase and allows for faster training or more layers in the network, as a result of which the performance of the network is improved.

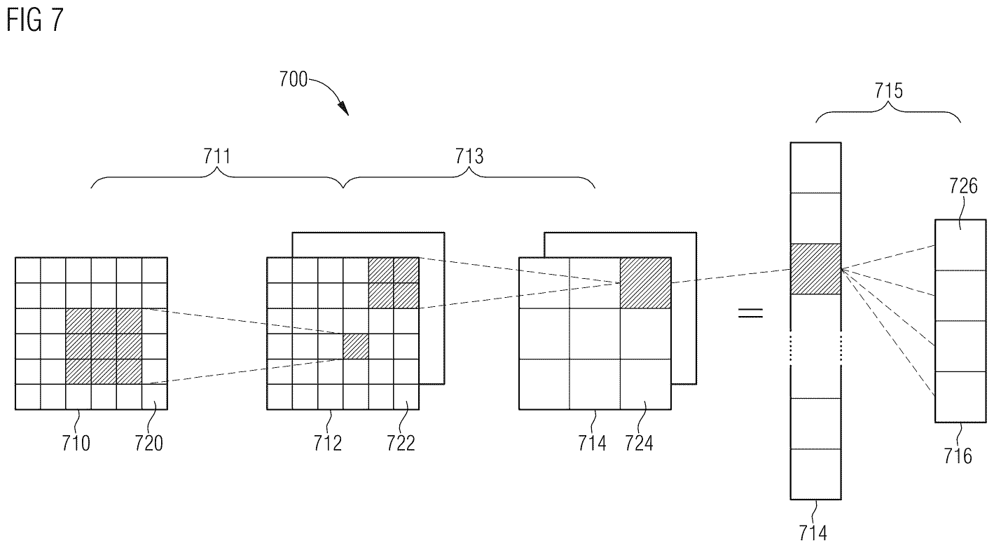

FIG. 7 shows an exemplary form of embodiment of a convolutional neural network 700. In the form of embodiment shown the convolutional neural network 700 comprises an input node layer 710, a convolution layer 711, a pooling layer 713, a fully connected layer 714, and an output node layer 716, as well as hidden node layers 712, 714. Alternatively, the convolutional neural network 700 can also comprise multiple convolution layers 711, multiple pooling layers 713 and/or multiple fully connected layers 715, as well as other types of layers. The sequence of the layers can be selected arbitrarily, with fully connected layers 715 generally being used as last layers prior to the output layer 716.

In particular, in a convolutional neural network 700 the nodes 720, 722, 724 of a node layer 710, 712, 714 can be regarded as a d-dimensional matrix or as a d-dimensional image. In particular, in the two-dimensional case the value of the node 720, 722, 724 indicated by i and j in the n-th node layer 710, 712, 714 can be referred to as x(n) [i, j]. However, the arrangement of the nodes 720, 722, 724 of a node layer 710, 712, 714 has as such no influence on the calculations which are performed within the convolutional neural network 700, since these are only given by the structure and the weights of the edges.

A convolution layer 711 is a connection layer between a front node layer 710 with node values x(n−1) and a rear node layer 712 with node values x(n). A convolution layer 711 is in particular characterized by the structure and the weights of the incoming edges, which form a convolution operation on the basis of a particular number of kernels. In particular, the structure and the weights of the edges of the convolution layer 711 are selected such that the values x(n) of the nodes 722 of the rear node layer 712 are calculated as a convolution x(n)=K*x(n−1) on the basis of the values x(n−1) of the nodes 720 of the front node layer 710, wherein the convolution * is defined in the two-dimensional case as

$${x}^{( n )} [ i , j ] = ( K * {x}^{( n - 1 )} ) [ i , j ] = {\sum }_{{i}^{′}} {\sum }_{{j}^{′}} K [ {i}^{′} , {j}^{′} ] \cdot {x}^{( n - 1 )} [ i - {i}^{′} , j - {j}^{′} ] .$$

In this case the kernel K is a d-dimensional matrix, in the present example a two-dimensional matrix, which generally is small in comparison to the number of nodes 720, 722, for example a 3×3 matrix or a 5×5 matrix. This means in particular that the weights of the edges in the convolution layer 711 are not independent, but are selected so that they produce the aforementioned convolution equation. In particular, for a kernel which is a 3×3 matrix, there are only 9 independent weights, wherein each entry in the kernel matrix corresponds to an independent weight, independent of the number of nodes 720, 722 in the front node layer 710 and the rear node layer 712.

In general, convolutional neural networks 700 use node layers 710, 712, 714 with a variety of channels, in particular due to the use of a variety of kernels in the convolution layers 711. In these cases, the node layers can be regarded as (d+1) dimensional matrices, wherein the first dimension indicates the channels. The effect of a convolution layer 711 is then defined in a two-dimensional example as

$${x}_{b}^{( n )} [ i , j ] = {\sum }_{a} ( {K}_{a , b} * {x}_{a}^{( n - 1 )} [ i , j ] = {\sum }_{a} {\sum }_{{i}^{′}} {\sum }_{{j}^{′}} {K}_{a , b} [ {i}^{′} , {j}^{′} ] \cdot {x}_{a}^{( n - 1 )} [ i - {i}^{′} , j - {j}^{′} ] ,$$

where

$${x}_{a}^{( n )}$$

corresponds to the a-th channel of the preceding node layer 710,

$${x}_{b}^{( n )}$$

corresponds to the b-th channel of the subsequent node layer 712 and K_{a,b} corresponds to one of the kernels. If a convolution layer 711 acts on a preceding node layer 710 with A channels and outputs a subsequent node layer 712 with B channels, there are A. B independent d-dimensional kernels K_{a,b}.

In general, activation functions can be used in convolutional neural networks 700. In this form of embodiment ReLU (rectified linear unit) is used, with R(z)=max(0, z), so that the effect of the convolution layer 711 in the two-dimensional example is

$${x}_{b}^{( n )} [ i , j ] = R ( {\sum }_{a} ( {K}_{a , b} * {x}_{a}^{( n - 1 )} [ i , j ] ) = R ( {\sum }_{a} {\sum }_{{i}^{′}} {\sum }_{{j}^{′}} {K}_{a , b} [ {i}^{′} , {j}^{′} ] \cdot {x}_{a}^{( n - 1 )} [ i - {i}^{′} , j - {j}^{′} ] )$$

It is also possible to use other activation functions, for example ELU (Exponential Linear Unit), LeakyReLU, Sigmoid, Tanh or Softmax.

In the form of embodiment displayed the input layer 710 contains 36 nodes 720, which are arranged in a two-dimensional 6×6 matrix. The first hidden node layer 712 contains 72 nodes 722, which are arranged as two-dimensional 6×6 matrices, wherein each of the two matrices is the result of a convolution of the values of the input layer with a 3×3 kernel within the convolution layer 711. Equivalent to this, the nodes 722 of the first hidden node layer 712 can be interpreted as a three-dimensional 2×6×6 matrix, wherein the first dimension corresponds to the channel dimension.

One advantage of using convolution layers 711 is that a spatially local correlation of the input data can be exploited by forcing a local connectivity pattern between the nodes of adjacent layers, in particular by connecting each node to only a small range of the nodes of the preceding layer.

A pooling layer 713 is a connection layer between a preceding node layer 712 with node values x(n−1) and a subsequent node layer 714 with node values x(n). A pooling layer 713 can in particular be characterized by the structure and the weights of the edges and the activation function, which form a pooling operation on the basis of a nonlinear pooling function f. For example, in the two-dimensional case the values x(n) of the nodes 724 of the subsequent node layer 714 can be calculated on the basis of the values x(n−1) of the nodes 722 of the anterior node layer 712 as follows

$${x}_{b}^{( n )} [ i , j ] = f ( {x}_{b}^{( n - 1 )} [ {id}_{1} , {jd}_{2} ] , … {x}_{b}^{( n - 1 )} [ ( i + 1 ) {d}_{1} - 1 , ( j + 1 ) {d}_{2} - 1 ] ) .$$

In other words by using a pooling layer 713 the number of nodes 722, 724 can be reduced, in that a number d1-d2 of adjacent nodes 722 in the preceding node layer 712 can be replaced by a single node 722 in the subsequent node layer 714, which is calculated as a function of the values of the aforementioned number of adjacent nodes. The pooling function f can in particular be the max function, the mean value or the L2 norm. In particular, in the case of a pooling layer 713 the weights of the incoming edges are fixed and are not changed by the training.

The advantage of using a pooling layer 713 is that the number of nodes 722, 724 and the number of parameters is reduced. This leads to a reduction in the amount of calculation work in the network and to a monitoring of over-adaptation.

In the form of embodiment shown the pooling layer 713 is a max pooling layer, in which four adjacent nodes are replaced by just one node, wherein the value is the maximum of the values of the four adjacent nodes. The max pooling is applied to each d-dimensional matrix of the preceding layer. In this form of embodiment the max pooling is applied to each of the two-dimensional matrices, as a result of which the number of nodes is reduced from 72 to 18.

In general the last layers of a convolutional neural network 700 can be fully connected layers 715. A fully connected layer 715 is a connection layer between a preceding node layer 714 and a subsequent node layer 716. A fully connected layer 713 can be characterized in that a majority of, in particular all, edges between the nodes 714 of the preceding node layer 714 and the nodes 716 of the subsequent node layer are present, and wherein the weight of each of these edges can be adjusted individually.

In this form of embodiment, the nodes 724 of the front node layer 714 of the fully connected layer 715 are represented both as two-dimensional matrices and additionally as non-contiguous nodes, which are displayed as a line of nodes, wherein the number of nodes has been reduced in the interest of clearer presentation. This procedure is also known as flattening. In this form of embodiment, the number of nodes 726 in the subsequent node layer 716 of the fully connected layer 715 is smaller than the number of nodes 724 in the preceding node layer 714. Alternatively, the number of nodes 726 can also be the same or larger.

Additionally, in this form of embodiment the Softmax activation function is used within the fully connected layer 715. By applying the Softmax function the sum of the values of all nodes 726 of the output layer 716 is equal to 1, and all values of all nodes 726 of the output layer 716 are real numbers between 0 and 1. In particular, when using the convolutional neural network 700 for categorizing input data the values of the output layer 716 can be interpreted as the probability that the input data falls into one of the various categories.

In particular, convolutional neural networks 700 can be trained on the basis of the backpropagation algorithm. To prevent over-adjustment, methods of regularization can be used, for example the omission of nodes 720, . . . , 724, stochastic pooling, the use of artificial data, weight decrease on the basis of the L1 or L2 norm or max norm restrictions.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A computer-implemented method for parameterizing an imaging system for mapping a clothed person, the computer-implemented method comprising:
obtaining image data about the clothed person;
determining body-shape information about the clothed person by applying a trained machine-learning model to the image data; and
determining at least one imaging parameter for the imaging system as a function of the body-shape information.

2. The computer-implemented method as claimed in claim 1, wherein the image data includes at least one of
a two-dimensional or two-and-a-half-dimensional image of the clothed person,
a video of the clothed person, or
a two-and-a-half-dimensional or three-dimensional point cloud, which represents the clothed person.

3. The computer-implemented method as claimed in claim 1, wherein the body-shape information at least one of,
describes a body contour of the clothed person, or
specifies respective positions of characteristic points of a body of the clothed person.

4. The computer-implemented method as claimed in claim 1, further comprising:
estimating secondary body information about the clothed person as a function of the body-shape information, and wherein
the at least one imaging parameter is determined as a function of the secondary body information.

5. The computer-implemented method as claimed in claim 4, wherein the secondary body information includes at least one of a body weight or a material composition of a body of the clothed person.

6. The computer-implemented method as claimed in claim 1, wherein the determining body-shape information comprises:
applying the trained machine-learning model to the image data to generate a body model of the clothed person; and
determining the body-shape information as a function of the body model.

7. The computer-implemented method as claimed in claim 1, wherein
the imaging system is an X-ray-based imaging system, and
the at least one imaging parameter includes at least one of
at least one exposure setting of an X-ray source of the imaging system,
a detector amplification of an X-ray detector of the imaging system,
a collimator position of a collimator of the imaging system,
a size of a collimator aperture of the collimator, or
a shape of the collimator aperture of the collimator; or
the imaging system is a magnetic resonance tomography system and the at least one imaging parameter includes a mapping target domain within a patient tube of the magnetic resonance tomography system.

8. The computer-implemented method as claimed in claim 1, wherein the imaging system is configured at least partially automatically in accordance with the at least one imaging parameter.

9. A computer-implemented training method for a machine-learning model for predicting body-shape information for a clothed person or a body model of the clothed person based on image data for the clothed person, the computer-implemented training method comprising:
obtaining training data; and
training an untrained or partially trained machine-learning model as a function of the training data, supervised or unsupervised, to (i) predict the body-shape information by applying the machine-learning model to the image data or (ii) predict the body model of the clothed person, from which the body-shape information is derivable.

10. The computer-implemented training method as claimed in claim 9, wherein
the machine-learning model includes a convolutional neural network or a transformer network and the training is supervised, and
the training data includes a variety of training datasets, wherein each of the training datasets includes training image data about a clothed person and associated basic truth data.

11. The computer-implemented training method as claimed in claim 9, wherein
the machine-learning model includes a generative adversarial network or a part of the generative adversarial network, and the training is unsupervised,
the training data includes a variety of first training datasets, wherein each of the first training datasets includes training image data about a clothed person, and
the training data includes a variety of second training datasets, wherein each of the second training datasets includes training body-shape information or a training body model of a person.

12. An imaging method for mapping a clothed person, the imaging method comprising:
determining at least one imaging parameter of an imaging system according to the computer-implemented method as claimed in claim 1; and
mapping the clothed person via the imaging system configured in accordance with the at least one imaging parameter.

13. The imaging method as claimed in claim 12, wherein at least one of
the image data about the clothed person is generated by a camera,
the imaging system is controlled to map the clothed person, or
a medical image dataset is generated by mapping the clothed person.

14. A data processing system configured to perform the computer-implemented method as claimed in claim 1.

15. An imaging apparatus comprising:
an imaging system configured to map a clothed person in accordance with at least one imaging parameter of the imaging system; and
a data processing system configured to perform the computer-implemented method as claimed in claim 1 to determine the at least one imaging parameter.

16. A non-transitory computer-readable storage medium storing computer-executable instructions that, when executed by a data processing system, cause the data processing system to perform the computer-implemented method as claimed in claim 1.

17. A data processing system configured to perform the computer-implemented training method as claimed in claim 9.

18. A non-transitory computer-readable storage medium storing computer-executable instructions that, when executed by a data processing system, cause the data processing system to perform the computer-implemented training method as claimed in claim 9.

19. A non-transitory computer-readable storage medium storing computer-executable instructions that, when executed by a data processing system at an imaging apparatus, cause the imaging apparatus to perform the imaging method as claimed in one claim 12.

20. The computer-implemented method as claimed in claim 2, further comprising:
estimating secondary body information about the clothed person as a function of the body-shape information, and wherein
the at least one imaging parameter is determined as a function of the secondary body information.
